(19) United States
(12) Patent Application Publication
(10) Pub. No.: US 2006/0182788 A1
(43) Pub. Date: Aug. 17, 2006

(54) Hydrophilic biocompatible adhesive formulations and uses

(76) Inventors: Parminder Singh, Union City, CA (US); Eun Soo Lee, Redwood City, CA (US); Appala Sagi, Redwood City, CA (US); Mikhail M. Feldstein, Moscow (RU); Danir F. Bairamov, Sunnyvale, CA (US)

Correspondence Address: MINTZ, LEVIN, COHN, FERRIS, GLOVSKY AND POPEO, P.C, 1400 PAGE MILL ROAD, PALO ALTO, CA, 94304-1124, US

(21) Appl. No.: 11/341,085

(22) Filed: Jan. 27, 2006

Related U.S. Application Data
(60) Provisional application No. 60/648,093, filed on Jan. 27, 2005.

(51) Int. Cl. A61F 13/02; A61L 15/16

(52) U.S. Cl. 424/448; 442/123

(57) ABSTRACT

This invention relates to the use of hydrophilic, biocompatible adhesives in drug delivery systems, wound dressings, bioelectrodes, and other systems in which hydrophilic, biocompatible adhesives are desirable. In particular, the invention relates to water-swellable, water-insoluble polymers that in combination render a composition adhesive upon contact with moisture, wherein a first water-swellable, water-insoluble polymer is cationic, a second water-swellable, water-insoluble polymer is anionic, and the polymers are ionically associated with each other to form a polymer matrix.

### CROSS-REFERENCE TO RELATED APPLICATION

This application claims priority under 35 U.S.C. § 119(e)(1) to Provisional U.S. Patent Application Ser. No. 60/648,093, filed Jan. 27, 2005, which is incorporated herein by reference in its entirety.

### TECHNICAL FIELD

This invention relates generally to hydrophilic, biocompatible adhesives. More specifically, it relates to the use of these adhesives in drug delivery systems, wound dressings, bioelectrodes, and other systems in which hydrophilic, biocompatible adhesives are desirable.

### BACKGROUND

Hydrophilic, biocompatible adhesives are well known for their various uses. Hydrophilic pressure-sensitive adhesives (“PSAs”) are used in a variety of pharmaceutical and cosmetic products, such as topical and transdermal drug delivery systems, wound dressings, bioelectrodes, face masks, bioadhesive films designed for buccal and mucosal administration, teeth whitening strips, and so on. A general distinctive feature of hydrophilic PSAs is that they typically adhere to wet biological substrates, while conventional hydrophobic (rubber-based) PSAs typically lose their adhesive properties when moistened.

Transdermal drug delivery systems generally include adhesives to hold the source of the drug on a body surface. The adhesive can cover the whole contact area, in which case it must be sufficiently permeable to allow movement of the drug through to the body surface. Alternatively, the adhesive can cover the edges of the system, excluding other substances from reaching the delivery area, but not participating directly in the drug delivery.

Transdermal drug delivery systems have a complicated set of requirements to meet in order to work successfully. They require adhesives with both high tack and an optimum slip-stick transition point. Drug release kinetics must be controlled with respect to delivery rate and the functional lifetime of the device. The device must be constructed so as to take into account specific characteristics of the drugs to be delivered: the device must be compatible with the drugs to be delivered, and must have the ability to store the drugs in a stable form. The devices must also be nontoxic and must not cause irritation or sensitization of the body surface to which they are applied. Such diverse requirements are difficult to combine in a single system.

Another use for these adhesives is in wound healing. Various types of bandages and wound dressings are known and used to protect wounds and burns. Typically, wound dressings are fabricated with an absorbent material so that wound exudate is removed and the wound dried, facilitating healing. Wound dressings may also contain one or more pharmacologically active agents such as antibiotics, local anesthetics, or the like. Commonly used wound dressings include fibrous materials such as gauze and cotton pads, which are advantageous in that they are absorbent but problematic in that fibers may adhere to the wound or newly forming tissue, causing wound injury upon removal. Other wound dressings have been prepared with foams and sponges, but the absorbance of these materials is often limited. Furthermore, such wound dressings require the use of adhesive tape, as they are not themselves adhesive.

To improve the absorbance of conventional fibrous wound dressings, water-swellable polymers, or “hydrogels,” have been incorporated into gauze or other fibrous materials for application to a wound. See, for example, U.S. Pat. No. 5,527,271 to Shah et al. However, the adhesion of fibers to the wound or newly forming tissue remains a significant disadvantage.

Another approach has been to use water-swellable polymeric materials instead of gauze, cotton, and the like. Wound-contacting surfaces made of such materials are not only more absorbent than conventional fibrous materials, they are also advantageous in that there is no risk of fiber adhesion during wound healing and upon removal of the wound dressing. Such wound dressings are disclosed, for example, in U.S. Pat. No. 4,867,748 to Samuelsen, which describes the use of an absorbent wound-contacting composition made from a water-soluble or water-swellable hydrocolloid blended with or dispersed in a water-insoluble, viscous, elastomeric binder. U.S. Pat. No. 6,201,164 to Wulff et al. describes a somewhat different type of hydrocolloid wound gel, consisting of a water-insoluble, water-swellable, crosslinked cellulose derivative, an alginate, and water.

Also used are hydrogel bandages, which are made from a liquid absorbing crosslinked polymer and have high water content prior to use. The high water content causes the hydrogel to exhibit very little or no adhesion, requiring the use of adhesive tape or a plaster such as 2^{nd} Skin® dressing available from Spenco Medical Ltd., U.K.

Another use for biocompatible gels is in medical diagnostics and treatments involving electricity. The composition may be used to attach a transcutaneous nerve stimulation electrode, an electrosurgical return electrode, or an EKG electrode, to a patient's skin or mucosal tissue. These applications involve modification of the composition so as to contain a conductive species. The form of attachment to the body surface must have minimal if any impedance of the electrical pulses being monitored. It must also adhere adequately to the body surface to allow reading of the electrical pulses from individual locations. It is also desirable that the contacts be easily removed from the body surface subsequent to testing and/or monitoring.

The adhesive properties of PSAs will vary depending upon how and where the products are to be used. For transdermal drug delivery and topical applications, an adhesive patch, for instance, should provide high tack immediately upon use, and such tack should be maintained during the entire application period (from one day to one week). For wound dressings and other various purposes, in order to avoid skin damage upon patch removal, adhesives that lose their adhesion under swelling in a large amount of water are preferred. Face masks work best using polymer matrices that adhere to the underlying tissue surface, but do not adhere to other surfaces.

Therefore, while the prior art discloses polymers and hydrogel compositions that can be tailored with respect to cohesive strength, adhesive strength, tack, elasticity, and water swellability, it remains desirable to develop appropriate compositions for drug delivery, wound healing, bioelectrodes, and the like.

### SUMMARY OF THE INVENTION

It is a primary object of the invention to provide compositions that address the above-mentioned needs in the art. In particular, polymer matrices are provided for pharmaceutical compositions for drug delivery, bioelectrodes, dressings to promote wound healing and the like that will adhere appropriately to a body surface to perform their function, while maintaining their cohesiveness.

In a first embodiment, a pharmaceutical composition is provided that comprises:

a therapeutically effective amount of an active agent; and

at least two water-swellable, water-insoluble polymers that in combination render the composition adhesive upon contact with moisture, wherein a first water-swellable, water-insoluble polymer is cationic, a second water-swellable, water-insoluble polymer is anionic, and the polymers are ionically associated with each other to form a polymer matrix. Preferably, at least one of the water-swellable, water-insoluble polymers is an acrylate-based polymer.

Optionally, the pharmaceutical composition is further comprised of 1.5 wt. % to 30 wt. % of a crosslinked hydrophilic polymer composition composed of (a) a covalently crosslinked hydrophilic polymer, and/or (b) a blend of a hydrophilic polymer and a complementary oligomer capable of hydrogen bonding thereto.

Preferred active agents include actives that function systemically or locally through transdermal delivery, and/or topically. Examples include, but are not limited to, analgesics, antibiotics, pain relievers, and vasodilators.

In another embodiment, a delivery system is provided for topical or transdermal administration of a pharmacologically active agent. The system is comprised of a laminated composite of:

a skin contact adhesive layer comprising the pharmaceutical composition as described above; and

laminated to the pharmaceutical composition, a flexible backing material that serves as the outer surface of the system following application to a body surface.

The delivery system may include a removable release liner covering the skin contact adhesive layer prior to use. This release liner prevents exposure of the adhesive layer to the air.

Further, the delivery system may include a nonwoven layer that bisects the skin contact adhesive layer. This nonwoven layer may assist in the manufacture of the system.

In a further embodiment, conductive bioadhesive compositions are provided that are comprised of:

at least two water-swellable, water-insoluble polymers that in combination render the composition adhesive upon contact with moisture, wherein a first water-swellable, water-insoluble polymer is cationic, a second water-swellable, water-insoluble polymer is anionic, and the polymers are ionically associated with each other to form a polymer matrix; and

an amount of an ionically conductive electrolyte effective to render the composition electrically conductive.

Preferably, at least one of the water-swellable, water-insoluble polymers is an acrylate-based polymer.

Such conductive bioadhesive compositions can be used for example in EKG and EEG tests, creating good adherence of test wires to relevant body parts while promoting the conductive flow of signals to the monitoring devices.

In yet another embodiment, a wound dressing is provided, which has a laminated composite of a body facing layer having a body-contacting surface, and an outwardly facing backing layer, wherein at least a portion of the body-contacting surface is composed of a water-swellable, water-insoluble polymer composition comprising at least two water-swellable, water-insoluble polymers that in combination render the composition adhesive upon contact with moisture, wherein a first water-swellable, water-insoluble polymer is cationic, a second water-swellable, water-insoluble polymer is anionic, and the polymers are ionically associated with each other to form a polymer matrix.

The wound dressing can further contain an active agent to assist in the healing of the wound, such as an antibiotic or a vasorestrictor.

Additionally, the body-facing layer of the wound dressing may have an inner region that contacts the wound and where adhesive is absent or decreased as compared to the outer edge of the body-facing layer.

### BRIEF DESCRIPTION OF THE FIGURE

FIG. 1 schematically illustrates a representative water-swellable, water-insoluble polymer system of the invention in the form of a laminated adhesive strip.

### DETAILED DESCRIPTION OF THE INVENTION

Before describing the present invention in detail, it is to be understood that unless otherwise indicated this invention is not limited to specific formulation materials or manufacturing processes, as such may vary. It is also to be understood that the terminology used herein is for the purpose of describing particular embodiments only, and is not intended to be limiting. It must be noted that, as used in this specification and the appended claims, the singular forms “a,” “an,” and “the” include plural referents unless the context clearly dictates otherwise. Thus, for example, reference to “a hydrophilic polymer” includes not only a single hydrophilic polymer but also a combination or mixture of two or more different hydrophilic polymers, reference to “a plasticizer” includes a combination or mixture of two or more different plasticizers as well as a single plasticizer, and the like.

In describing and claiming the present invention, the following terminology will be used in accordance with the definitions set out below.

The definitions of “hydrophobic” and “hydrophilic” polymers are based on the amount of water vapor absorbed by polymers at 100% relative humidity. According to this classification, hydrophobic polymers absorb only up to 1 wt. % water at 100% relative humidity (“rh”), while moderately hydrophilic polymers absorb 1-10% wt. % water, hydrophilic polymers are capable of absorbing more than 10 wt. % of water, and hygroscopic polymers absorb more than 20 wt. % of water. A “water-swellable” polymer is one that absorbs an amount of water greater than at least 50 wt. % of its own weight, upon immersion in an aqueous medium.

The term “crosslinked” herein refers to a composition containing intramolecular and/or intermolecular crosslinks, whether arising through covalent or noncovalent bonding. “Noncovalent” bonding includes hydrogen bonding, ionic bonding, and electrostatic bonding.

The term “polymer” includes linear and branched polymer structures, and also encompasses crosslinked polymers as well as copolymers (which may or may not be crosslinked), thus including block copolymers, alternating copolymers, random copolymers, and the like. Those compounds referred to herein as “oligomers” are polymers having a molecular weight below about 1000 Da, preferably below about 800 Da.

In a first embodiment, a pharmaceutical composition is provided, having an admixture of a therapeutically effective amount of an active agent and at least two water-swellable, water-insoluble polymers that in combination render the composition adhesive upon contact with moisture. Among the water-swellable, water-insoluble polymers, a first water-swellable, water-insoluble polymer is cationic, and a second water-swellable, water-insoluble polymer is anionic, and the polymers are ionically associated with each other to form a polymer matrix. In a preferred embodiment, the composition is composed of two water-swellable, water-insoluble polymers, one cationic and the other anionic.

The water-swellable, water-insoluble polymers are capable of at least some degree of swelling when immersed in an aqueous liquid but are either completely insoluble in water or water insoluble within a selected pH range, e.g., a pH in the range of about 7.0 to about 8.5.

Preferably, at least one of the water-swellable, water-insoluble polymers of the composition is an acrylate-based polymer. Alternatively, all water-swellable, water-insoluble polymers in the composition are acrylate-based polymers.

Preferred water-swellable polymers include, but are not limited to, acrylate polymers, generally formed from acrylic acid, methacrylic acid, acrylate, methyl acrylate, ethyl acrylate, methyl methacrylate, ethyl methacrylate, a dialkylaminoalkyl acrylate, a dialkylaminoalkyl methacrylate, a trialkylammonioalkyl acrylate, and/or a trialkylammonioalkyl methacrylate. Preferred are polymers or copolymers of acrylic acid, methacrylic acid, methyl methacrylate, ethyl methacrylate, 2-dimethylaminoethyl methacrylate, and trimethylammonioethyl methacrylate chloride.

Suitable acrylate polymers are those copolymers available under the tradename “Eudragit” from Rohm Pharma (Germany). The Eudragit series E, L, S, RL, RS and NE copolymers are available as solubilized in organic solvent, in an aqueous dispersion, or as a dry powder. Preferred acrylate polymers are copolymers of methacrylic acid and methyl methacrylate, such as the Eudragit L and Eudragit S series polymers. Particularly preferred such copolymers are Eudragit L-30D-55 and Eudragit L-100-55 (the latter copolymer is a spray-dried form of Eudragit L-30D-55 that can be reconstituted with water). The molecular weight of the Eudragit L-30D-55 and Eudragit L-100-55 copolymer is approximately 135,000 Da, with a ratio of free carboxyl groups to ester groups of approximately 1:1. The copolymer is generally insoluble in aqueous fluids having a pH below 5.5. Another particularly suitable methacrylic acid-methyl methacrylate copolymer is Eudragit S-100, which differs from Eudragit L-30D-55 in that the ratio of free carboxyl groups to ester groups is approximately 1:2. Eudragit S-100 is insoluble at pH below 5.5, but unlike Eudragit L-30D-55, is poorly soluble in aqueous fluids having a pH in the range of 5.5 to 7.0. This copolymer is soluble at pH 7.0 and above. Eudragit L-100 may also be used, which has a pH-dependent solubility profile between that of Eudragit L-30D-55 and Eudragit S-100, insofar as it is insoluble at a pH below 6.0. It will be appreciated by those skilled in the art that Eudragit L-30D-55, L-100-55, L-100, and S-100 can be replaced with other acceptable polymers having similar pH-dependent solubility characteristics.

Other preferred acrylate polymers are cationic, such as the Eudragit E, RS, and RL series polymers. Eudragit E100 and E PO are cationic copolymers of dimethylaminoethyl methacrylate and neutral methacrylates (e.g., methyl methacrylate), while Eudragit RS and Eudragit RL polymers are analogous polymers, composed of neutral methacrylic acid esters and a small proportion of trimethylammonioethyl methacrylate.

In one embodiment of the invention, the cationic polymer may be an acrylate-based polymer with pendant quaternary ammonium groups or tertiary amino groups (as exemplified by a Eudragit RS, Eudragit RL, Eudragit E copolymer), and the anionic polymer may be an ionized acrylic acid or methacrylic acid polymer such as a Eudragit L or Eudragit S copolymer.

In a preferred embodiment, a crosslinked hydrophilic polymer composition is incorporated into the composition. The crosslinked hydrophilic polymer composition may be composed of (a) a covalently crosslinked hydrophilic polymer, and/or (b) a blend of a hydrophilic polymer and a complementary oligomer capable of hydrogen bonding thereto.

Suitable hydrophilic polymers include repeating units derived from an N-vinyl lactam monomer, a carboxy vinyl monomer, a vinyl ester monomer, an ester of a carboxy vinyl monomer, a vinyl amide monomer, and/or a hydroxy vinyl monomer. Such polymers include, by way of example, poly(N-vinyl lactams), poly(N-vinyl acrylamides), poly(N-alkylacrylamides), substituted and unsubstituted acrylic and methacrylic acid polymers, polyvinyl alcohol (PVA), polyvinylamine, copolymers thereof and copolymers with other types of hydrophilic monomers (e.g. vinyl acetate). Other suitable hydrophilic polymers include, but are not limited to: polysaccharides; crosslinked acrylate polymers and copolymers; carbomers, i.e., hydroxylated vinylic polymers also referred to as “interpolymers,” which are prepared by crosslinking a monoolefinic acrylic acid monomer with a polyalkyl ether of sucrose (commercially available under the trademark Carbopol® from the B.F. Goodrich Chemical Company); crosslinked acrylamide-sodium acrylate copolymers; gelatin; vegetable polysaccharides, such as alginates, pectins, carrageenans, or xanthan; starch and starch derivatives; and galactomannan and galactomannan derivatives.

Polysaccharide materials include, for instance, crosslinked, normally water-soluble cellulose derivatives that are crosslinked to provide water-insoluble, water-swellable compounds, such as crosslinked sodium carboxymethylcellulose (CMC), crosslinked hydroxyethyl cellulose (HEC), crosslinked partial free acid CMC, and guar gum grafted with acrylamide and acrylic acid salts in combination with divinyl compounds, e.g., methylene-bis acrylamide. Within the aforementioned class, the more preferred materials are crosslinked CMC derivatives, particularly crosslinked sodium CMC and crosslinked HEC. Other polysaccharides suitable herein include hydroxypropyl cellulose (HPC), hydroxypropyl methylcellulose (HPMC), hydroxypropyl cellulose (HPC), and the like.

Poly(N-vinyl lactams) useful herein are preferably homopolymers or copolymers of N-vinyl lactam monomer units, with N-vinyl lactam monomer units representing the majority of the total monomeric units of a poly(N-vinyl lactams) copolymer. Preferred poly(N-vinyl lactams) for use in conjunction with the invention are prepared by polymerization of one or more of the following N-vinyl lactam monomers: N-vinyl-2-pyrrolidone; N-vinyl-2-valerolactam; and N-vinyl-2-caprolactam. Nonlimiting examples of non-N-vinyl lactam comonomers useful with N-vinyl lactam monomeric units include N,N-dimethylacrylamide, acrylic acid, methacrylic acid, hydroxyethyl methacrylate, acrylamide, 2-acrylamido-2-methyl-1-propane sulfonic acid or its salt, and vinyl acetate.

Poly(N-alkylacrylamides) include, by way of example, poly(methacrylamide) and poly(N-isopropyl acrylamide) (PNIPAM). Polymers of carboxy vinyl monomers are typically formed from acrylic acid, methacrylic acid, crotonic acid, isocrotonic acid, itaconic acid and anhydride, a 1,2-dicarboxylic acid such as maleic acid or fumaric acid, maleic anhydride, or mixtures thereof, with preferred hydrophilic polymers within this class including polyacrylic acid and polymethacrylic acid, with polyacrylic acid most preferred.

Preferred hydrophilic polymers herein are the following: poly(N-vinyl lactams), particularly polyvinyl pyrrolidone (PVP) and poly(N-vinyl caprolactam) (PVCap); poly(N-vinyl acetamides), particularly polyacetamide per se; polymers of carboxy vinyl monomers, particularly polyacrylic acid and polymethacrylic acid; and copolymers and blends thereof. PVP and PVCap are particularly preferred.

The molecular weight of the hydrophilic polymer is not critical; however, the number average molecular weight of the hydrophilic polymer is generally in the range of approximately 20,000 to 2,000,000, more typically in the range of approximately 200,000 to 1,000,000.

Covalent crosslinking may be accomplished in several ways. For instance, the hydrophilic polymer, or the hydrophilic polymer and a complementary oligomer, may be covalently crosslinked using heat, radiation, or a chemical curing (crosslinking) agent. Covalently crosslinked hydrophilic polymers may also be obtained commercially, for example, crosslinked sodium CMC is available under the tradename Aquasorb® (e.g., Aquasorb® A500) from Aqualon, a division of Hercules, Inc., and crosslinked PVP is available under the tradename Kollidon® (e.g., Kollidon® CL, and Kollidon® CL-M, a micronized form of crosslinked PVP, both available from BASF).

For thermal crosslinking, a free radical polymerization initiator is used, and can be any of the known free radical-generating initiators conventionally used in vinyl polymerization. Preferred initiators are organic peroxides and azo compounds, generally used in an amount from about 0.01 wt. % to 15 wt. %, preferably 0.05 wt. % to 10 wt. %, more preferably from about 0.1 wt. % to about 5% and most preferably from about 0.5 wt. % to about 4 wt. % of the polymerizable material. Suitable organic peroxides include dialkyl peroxides such as t-butyl peroxide and 2,2 bis(t-butylperoxy)propane, diacyl peroxides such as benzoyl peroxide and acetyl peroxide, peresters such as t-butyl perbenzoate and t-butyl per-2-ethylhexanoate, perdicarbonates such as dicetyl peroxy dicarbonate and dicyclohexyl peroxy dicarbonate, ketone peroxides such as cyclohexanone peroxide and methylethylketone peroxide, and hydroperoxides such as cumene hydroperoxide and tert-butyl hydroperoxide. Suitable azo compounds include azo bis(isobutyronitrile) and azo bis(2,4-dimethylvaleronitrile). The temperature for thermal crosslinking will depend on the actual components and may be readily deduced by one of ordinary skill in the art, but typically ranges from about 80° C. to about 200° C.

Crosslinking may also be accomplished with radiation, typically in the presence of a photoinitator. The radiation may be ultraviolet, alpha, beta, gamma, electron beam, and x-ray radiation, although ultraviolet radiation is preferred. Useful photosensitizers are triplet sensitizers of the “hydrogen abstraction” type, and include benzophenone and substituted benzophenone and acetophenones such as benzyl dimethyl ketal, 4-acryloxybenzophenone (ABP), 1-hydroxy-cyclohexyl phenyl ketone, 2,2-diethoxyacetophenone and 2,2-dimethoxy-2-phenylaceto-phenone, substituted alpha-ketols such as 2-methyl-2-hydroxypropiophenone, benzoin ethers such as benzoin methyl ether and benzoin isopropyl ether, substituted benzoin ethers such as anisoin methyl ether, aromatic sulfonyl chlorides such as 2-naphthalene sulfonyl chloride, photoactive oximes such as 1-phenyl-1,2-propanedione-2-(O-ethoxy-carbonyl)-oxime, thioxanthones including alkyl- and halogen-substituted thioxanthones such as 2-isopropylthioxanthone, 2-chlorothioxanthone, 2,4 dimethyl thioxanone, 2,4 dichlorothioxanone, and 2,4-diethyl thioxanone, and acyl phosphine oxides. Radiation having a wavelength of 200 to 800 nm, preferably, 200 to 500 nm, is preferred for use herein, and low intensity ultraviolet light is sufficient to induce crosslinking in most cases. However, with photosensitizers of the hydrogen abstraction type, higher intensity UV exposure may be necessary to achieve sufficient crosslinking. Such exposure can be provided by a mercury lamp processor such as those available from PPG, Fusion, Xenon, and others. Crosslinking may also be induced by irradiating with gamma radiation or an electron beam. Appropriate irradiation parameters, i.e., the type and dose of radiation used to effect crosslinking, will be apparent to those skilled in the art.

Suitable chemical curing agents, also referred to as chemical cross-linking “promoters,” include, without limitation, polymercaptans such as 2,2-dimercapto diethylether, dipentaerythritol hexa(3-mercaptopropionate), ethylene bis(3-mercaptoacetate), pentaerythritol tetra(3-mercaptopropionate), pentaerythritol tetrathioglycolate, polyethylene glycol dimercaptoacetate, polyethylene glycol di(3-mercaptopropionate), trimethylolethane tri(3-mercaptopropionate), trimethylolethane trithioglycolate, trimethylolpropane tri(3-mercapto-propionate), trimethylolpropane trithioglycolate, dithioethane, di- or trithiopropane and 1,6-hexane dithiol. The crosslinking promoter is added to the uncrosslinked hydrophilic polymer to promote covalent crosslinking thereof, or to a blend of the uncrosslinked hydrophilic polymer and the complementary oligomer, to provide crosslinking between the two components.

The crosslinked hydrophilic polymer may also comprise a blend of a hydrophilic polymer and a low molecular weight complementary oligomer capable of crosslinking the polymer via hydrogen bonding. In this case, the hydrophilic polymer may or may not be crosslinked prior to admixture with the complementary oligomer. If the hydrophilic polymer is crosslinked prior to admixture with the complementary oligomer, it may be preferred to synthesize the polymer in crosslinked form, by admixing a monomeric precursor to the polymer with multifunctional comonomer and copolymerizing. Examples of monomeric precursors and corresponding polymeric products are as follows: N-vinyl amide precursors for a poly(N-vinyl amide) product; N-alkylacrylamides for a poly(N-alkylacrylamide) product; acrylic acid for a polyacrylic acid product; methacrylic acid for a polymethacrylic acid product; acrylonitrile for a poly(acrylonitrile) product; and N-vinyl pyrrolidone (NVP) for a poly(vinylpyrrolidone) (PVP) product. Polymerization may be carried out in bulk, in suspension, in solution, or in an emulsion. Solution polymerization is preferred, and polar organic solvents such as ethyl acetate and lower alkanols (e.g., ethanol, isopropyl alcohol, etc.) are particularly preferred. For preparation of hydrophilic vinyl polymers, synthesis will typically take place via a free radical polymerization process in the presence of a free radical initiator as described above. The multifunctional comonomer include, for example, bisacrylamide, acrylic or methacrylic esters of diols such as butanediol and hexanediol (1,6-hexane diol diacrylate is preferred), other acrylates such as pentaerythritol tetraacrylate, and 1,2-ethylene glycol diacrylate, and 1,12-dodecanediol diacrylate. Other useful multifunctional crosslinking monomers include oligomeric and polymeric multifunctional (meth)acrylates, e.g., poly(ethylene oxide) diacrylate or poly(ethylene oxide) dimethacrylate; polyvinylic crosslinking agents such as substituted and unsubstituted divinylbenzene; and difunctional urethane acrylates such as EBECRYL® 270 and EBECRYL® 230 (1500 weight average molecular weight and 5000 weight average molecular weight acrylated urethanes, respectively—both available from UCB of Smyrna, Ga.), and combinations thereof. If a chemical crosslinking agent is employed, the amount used will preferably be such that the weight ratio of crosslinking agent to hydrophilic polymer is in the range of about 1:100 to 1:5. To achieve a higher crosslink density, if desired, chemical crosslinking is combined with radiation curing.

If the crosslinked hydrophilic polymer is in the form of a blend of a hydrophilic polymer and a low molecular weight complementary oligomer, the blend will usually provide a matrix that is crosslinked solely by hydrogen bonds formed between the termini of the oligomer and pendant groups on the hydrophilic polymer. In this embodiment, suitable hydrophilic polymers include repeating units derived from an N-vinyl lactam monomer, a carboxy vinyl monomer, a vinyl ester monomer, an ester of a carboxy vinyl monomer, a vinyl amide monomer, and/or a hydroxy vinyl monomer, as described above with regard to crosslinked hydrophilic polymers per se, and preferred hydrophilic polymers in this blend are also as described above for those polymers.

The oligomer that is “complementary” to the hydrophilic polymer in that it is capable of hydrogen bonding thereto. Preferably, the complementary oligomer is terminated with hydroxyl groups, amino groups, or carboxyl groups. The oligomer typically has a glass transition temperature T_{g} in the range of about −100° C. to about −30° C. and a melting temperature Tₘ lower than about 20° C. The oligomer may be also amorphous. The difference between the T_{g} values the hydrophilic polymer and the oligomer is preferably greater than about 50° C., more preferably greater than about 100° C., and most preferably in the range of about 150° C. to about 300° C. The hydrophilic polymer and complementary oligomer should be compatible, i.e. capable of forming a homogeneous blend that exhibits a single T_{g}, intermediate between those of the unblended components. Generally, the oligomer will have a molecular weight in the range from about 45 to about 800, preferably in the range of about 45 to about 600. Examples of suitable oligomers include, but are not limited to, low molecular weight polyalcohols (e.g. glycerol), oligoalkylene glycols such as ethylene glycol and propylene glycol, ether alcohols (e.g., glycol ethers), alkane diols from butane diol to octane diol, including carboxyl-terminated and amino-terminated derivatives of polyalkylene glycols. Polyalkylene glycols, optionally carboxyl-terminated, are preferred herein, and polyethylene glycol having a molecular weight in the range of about 300 to 600 is an optimal complementary oligomer.

The hydrophilic polymer and the complementary oligomer should be miscible with respect to each other and have disparate chain lengths (as may be deduced from the above). The ratio of the weight average molecular weight of the hydrophilic polymer to that of the oligomer should be within about 200 and 200,000, preferably within about 1,250 and 20,000. Also, the polymer and the oligomer should contain complementary functional groups capable of hydrogen bonding, ionic bonding, electrostatic bonding, or covalent bonding to each other. Ideally, the complementary functional groups of the polymer are located throughout the polymeric structure, while the functional groups of the oligomer are preferably located at the two termini of a linear molecule, and are not present along the backbone. Forming hydrogen bonds or ionic bonds between the two terminal functional groups of the oligomer and the corresponding functional groups contained along the backbone of the hydrophilic polymer results in a noncovalently linked supramolecular network.

As discussed in U.S. Pat. No. 6,576,712 to Feldstein et al., the ratio of the hydrophilic polymer to the complementary oligomer in the aforementioned blend affects both adhesive strength and cohesive strength. As explained in the aforementioned patent, the complementary oligomer decreases the glass transition of the hydrophilic polymer/complementary oligomer blend to a greater degree than predicted by the Fox equation, which is given by equation (1)

$$\frac{1}{{T}_{g predicted}} = \frac{{w}_{pol}}{{T}_{g pol}} + \frac{{w}_{pl}}{{T}_{g pl}} \qquad ( 1 )$$

where T_{g predicted} is the predicted glass transition temperature of the hydrophilic polymer/complementary oligomer blend, wₚₒₗ is the weight fraction of the hydrophilic polymer in the blend, wₚₗ is the weight fraction of the complementary oligomer in the blend, T_{g pol} is the glass transition temperature of the hydrophilic polymer, and T_{g pl} is the glass transition temperature of the complementary oligomer. As also explained in that patent, an adhesive composition having optimized adhesive and cohesive strength can be prepared from a hydrophilic polymer and a complementary oligomer by selecting the components and their relative amounts to give a predetermined deviation from T_{g predicted}. Generally, to maximize adhesion, the predetermined deviation from T_{g predicted} will be the maximum negative deviation, while to minimize adhesion, any negative deviation from T_{g predicted} is minimized. Optimally, the complementary oligomer represents approximately 25 wt. % to 75 wt. %, preferably about 30 wt. % to about 60 wt. %, of the hydrophilic polymer/complementary oligomer blend, and, correspondingly, the hydrophilic polymer represents approximately 75 wt. % to 25 wt. %, preferably about 70 wt. % to about 40 wt. %, of the hydrophilic polymer/oligomer blend.

For certain applications, the hydrophilic polymer and optionally the complementary oligomer may be covalently crosslinked. The hydrophilic polymer may be covalently crosslinked, either intramolecularly or intermolecularly, and/or the hydrophilic polymer and the complementary oligomer may be covalently crosslinked. In the former case, there are no covalent bonds linking the hydrophilic polymer to the complementary oligomer, while in the latter case, there are covalent crosslinks binding the hydrophilic polymer to the complementary oligomer. The hydrophilic polymer, or the hydrophilic polymer and the complementary oligomer, may be covalently crosslinked using heat, radiation, or a chemical curing (crosslinking) agent. The degree of crosslinking should be sufficient to eliminate or at least minimize cold flow under compression.

For covalently crosslinked hydrophilic polymer/complementary oligomer systems, the oligomer should be terminated at each end with a group capable of undergoing reaction with a functional group on the hydrophilic polymer. Such reactive groups include, for example, hydroxyl groups, amino groups, and carboxyl groups. These difunctionalized oligomers may be obtained commercially or readily synthesized using techniques known to those of ordinary skill in the art and/or described in the pertinent texts and literature.

As the complementary oligomer may itself act as a plasticizer, it is not generally necessary to incorporate an added low molecular weight plasticizer into the present compositions unless the optional complementary oligomer is not included. Suitable low molecular weight plasticizers include: dialkyl phthalates, dicycloalkyl phthalates, diaryl phthalates, and mixed alkyl-aryl phthalates, as represented by dimethyl phthalate, diethyl phthalate, dipropyl phthalate, di(2-ethylhexyl)-phthalate, di-isopropyl phthalate, diamyl phthalate and dicapryl phthalate; alkyl and aryl phosphates such as tributyl phosphate, trioctyl phosphate, tricresyl phosphate, and triphenyl phosphate; alkyl citrate and citrate esters such as trimethyl citrate, triethyl citrate, tributyl citrate, acetyl triethyl citrate, and trihexyl citrate; dialkyl adipates such as dioctyl adipate (DOA); also referred to as bis(2-ethylhexyl)adipate), diethyl adipate, di(2-methylethyl)adipate, and dihexyl adipate; dialkyl tartrates such as diethyl tartrate and dibutyl tartrate; dialkyl sebacates such as diethyl sebacate, dipropyl sebacate and dinonyl sebacate; dialkyl succinates such as diethyl succinate and dibutyl succinate; alkyl glycolates, alkyl glycerolates, glycol esters and glycerol esters such as glycerol diacetate, glycerol triacetate (triacetin), glycerol monolactate diacetate, methyl phthalyl ethyl glycolate, butyl phthalyl butyl glycolate, ethylene glycol diacetate, ethylene glycol dibutyrate, triethylene glycol diacetate, triethylene glycol dibutyrate and triethylene glycol dipropionate; and mixtures thereof. Preferred low molecular weight plasticizers for the continuous hydrophilic phase are triethyl citrate, diethyl phthalate, and dioctyl adipate, with dioctyl adipate most preferred.

The properties of the compositions of the invention are readily controlled by adjusting one or more parameters during formulation. For example, the adhesiveness of the composition can be controlled during manufacture in order to increase or decrease the degree to which the composition will adhere to a body surface in the presence of moisture. This can be accomplished by varying type and/or amount of different components, or by changing the mode of manufacture. Also, with respect to the fabrication process, compositions prepared using a conventional melt extrusion process are generally, although not necessarily, somewhat less tacky than compositions prepared using a solution cast technique.

Active Agents

Suitable active agents that may be incorporated into the present pharmaceutical compositions and delivered systemically (e.g., with a transdermal, oral, or other dosage form suitable for systemic administration of a drug) include, but are not limited to: analeptic agents; analgesic agents; anesthetic agents; antiarthritic agents; respiratory drugs, including antiasthmatic agents; anticancer agents, including antineoplastic drugs; anticholinergics; anticonvulsants; antidepressants; antidiabetic agents; antidiarrheals; antihelminthics; antihistamines; antihyperlipidemic agents; antihypertensive agents; anti-infective agents such as antibiotics and antiviral agents; antiinflammatory agents; antimigraine preparations; antinauseants; antiparkinsonism drugs; antipruritics; antipsychotics; antipyretics; antispasmodics; antitubercular agents; antiulcer agents; antiviral agents; anxiolytics; appetite suppressants; attention deficit disorder (ADD) and attention deficit hyperactivity disorder (ADHD) drugs; cardiovascular preparations including calcium channel blockers, antianginal agents, central nervous system (CNS) agents, beta-blockers and antiarrhythmic agents; central nervous system stimulants; cough and cold preparations, including decongestants; diuretics; genetic materials; herbal remedies; hormonolytics; hypnotics; hypoglycemic agents; immunosuppressive agents; leukotriene inhibitors; mitotic inhibitors; muscle relaxants; narcotic antagonists; nicotine; nutritional agents, such as vitamins, essential amino acids and fatty acids; ophthalmic drugs such as antiglaucoma agents; parasympatholytics; peptide drugs; psychostimulants; sedatives; steroids, including progestogens, estrogens, corticosteroids, androgens and anabolic agents; smoking cessation agents; sympathomimetics; tranquilizers; and vasodilators including general coronary, peripheral and cerebral. Specific active agents with which the present adhesive compositions are useful include, without limitation, anabasine, capsaicin, isosorbide dinitrate, aminostigmine, nitroglycerine, verapamil, propranolol, silabolin, foridone, clonidine, cytisine, phenazepam, nifedipine, fluacizin, and salbutamol.

For topical drug administration, suitable active agents include, by way of example, the following:

Bacteriostatic and bactericidal agents: Suitable bacteriostatic and bactericidal agents include, by way of example: halogen compounds such as iodine, iodopovidone complexes (i.e., complexes of PVP and iodine, also referred to as “povidine” and available under the tradename Betadine® from Purdue Frederick), iodide salts, chloramine, chlorohexidine, and sodium hypochlorite; silver and silver-containing compounds such as sulfadiazine, silver protein acetyltannate, silver nitrate, silver acetate, silver lactate, silver sulfate and silver chloride; organotin compounds such as tri-n-butyltin benzoate; zinc and zinc salts; oxidants, such as hydrogen peroxide and potassium permanganate; aryl mercury compounds, such as phenylmercury borate or merbromin; alkyl mercury compounds, such as thiomersal; phenols, such as thymol, o-phenyl phenol, 2-benzyl-4-chlorophenol, hexachlorophen and hexylresorcinol; and organic nitrogen compounds such as 8-hydroxyquinoline, chlorquinaldol, clioquinol, ethacridine, hexetidine, chlorhexedine, and ambazone.

Antibiotic agents: Suitable antibiotic agents include, but are not limited to, antibiotics of the lincomycin family (referring to a class of antibiotic agents originally recovered from Streptomyces lincolnensis), antibiotics of the tetracycline family (referring to a class of antibiotic agents originally recovered from Streptomyces aureofaciens), and sulfur-based antibiotics, i.e., sulfonamides. Exemplary antibiotics of the lincomycin family include lincomycin itself (6,8-dideoxy-6-[[(1-methyl-4-propyl-2-pyrrolidinyl)-carbonyl]amino]-1-thio-L-threo-α-D-galactooctopyranoside), clindamycin, the 7-deoxy, 7-chloro derivative of lincomycin (i.e., 7-chloro-6,7,8-trideoxy-6-[[(1-methyl-4-propyl-2-pyrrolidinyl)carbonyl]amino]-1-thio-L-threo-α-D-galacto-octopyranoside), related compounds as described, for example, in U.S. Pat. Nos. 3,475,407, 3,509,127, 3,544,551 and 3,513,155, and pharmacologically acceptable salts and esters thereof. Exemplary antibiotics of the tetracycline family include tetracycline itself, 4-(dimethylamino)-1,4,4α,5,5α,6,11,12αoctahydro3,6,12,12α-pentahydroxy-6-methyl-1,11-dioxo-2-naphthacenecarboxamide), chlortetracycline, oxytetracycline, tetracycline, demeclocycline, rolitetracycline, methacycline and doxycycline and their pharmaceutically acceptable salts and esters, particularly acid addition salts such as the hydrochloride salt. Exemplary sulfur-based antibiotics include, but are not limited to, the sulfonamides sulfacetamide, sulfabenzamide, sulfadiazine, sulfadoxine, sulfamerazine, sulfamethazine, sulfamethizole, sulfamethoxazole, and pharmacologically acceptable salts and esters thereof, e.g., sulfacetamide sodium.

Pain relieving agents: Suitable pain relieving agents are local anesthetics, including, but not limited to, acetamidoeugenol, alfadolone acetate, alfaxalone, amucaine, amolanone, amylocalne, benoxinate, betoxycaine, biphenamine, bupivacaine, burethamine, butacaine, butaben, butanilicaine, buthalital, butoxycaine, carticaine, 2-chloroprocaine, cinchocaine, cocaethylene, cocaine, cyclomethycaine, dibucaine, dimethisoquin, dimethocaine, diperadon, dyclonine, ecgonidine, ecgonine, ethyl aminobenzoate, ethyl chloride, etidocaine, etoxadrol, β-eucaine, euprocin, fenalcomine, fomocaine, hexobarbital, hexylcaine, hydroxydione, hydroxyprocaine, hydroxytetracaine, isobutyl p-aminobenzoate, kentamine, leucinocaine mesylate, levoxadrol, lidocaine, mepivacaine, meprylcaine, metabutoxycaine, methohexital, methyl chloride, midazolam, myrtecaine, naepaine, octacaine, orthocaine, oxethazaine, parethoxycaine, phenacaine, phencyclidine, phenol, piperocaine, piridocaine, polidocanol, pramoxine, prilocalne, procaine, propanidid, propanocaine, proparacaine, propipocaine, propofol, propoxycaine, pseudococaine, pyrrocaine, risocaine, salicyl alcohol, tetracaine, thialbarbital, thimylal, thiobutabarbital, thiopental, tolycaine, trimecaine, zolamine, and combinations thereof. Tetracaine, lidocaine and prilocalne are referred pain relieving agents herein.

Other topical agents that may be delivered using the present compositions as drug delivery systems include the following: antifungal agents such as undecylenic acid, tolnaftate, miconazole, griseofulvine, ketoconazole, ciclopirox, clotrimazole and chloroxylenol; keratolytic agents, such as salicylic acid, lactic acid and urea; vessicants such as cantharidin; anti-acne agents such as organic peroxides (e.g., benzoyl peroxide), retinoids (e.g., retinoic acid, adapalene, and tazarotene), sulfonamides (e.g., sodium sulfacetamide), resorcinol, corticosteroids (e.g., triamcinolone), alpha-hydroxy acids (e.g., lactic acid and glycolic acid), alpha-keto acids (e.g., glyoxylic acid), and antibacterial agents specifically indicated for the treatment of acne, including azelaic acid, clindamycin, erythromycin, meclocycline, minocycline, nadifloxacin, cephalexin, doxycycline, and ofloxacin; skin-lightening and bleaching agents, such as hydroquinone, kojic acid, glycolic acid and other alpha-hydroxy acids, artocarpin, and certain organic peroxides; agents for treating warts, including salicylic acid, imiquimod, dinitrochlorobenzene, dibutyl squaric acid, podophyllin, podophyllotoxin, cantharidin, trichloroacetic acid, bleomycin, cidofovir, adefovir, and analogs thereof; and anti-inflammatory agents such as corticosteroids and nonsteroidal anti-inflammatory drugs (NSAIDs), where the NSAIDS include ketoprofen, flurbiprofen, ibuprofen, naproxen, fenoprofen, benoxaprofen, indoprofen, pirprofen, carprofen, oxaprozin, pranoprofen, suprofen, alminoprofen, butibufen, fenbufen, and tiaprofenic acid.

For wound dressings, suitable active agents are those useful for the treatment of wounds, and include, but are not limited to bacteriostatic and bactericidal compounds, antibiotic agents, pain relieving agents, vasodilators, tissue-healing enhancing agents, amino acids, proteins, proteolytic enzymes, cytokines, and polypeptide growth factors. Specific such agents are set forth below.

For topical and transdermal administration of some active agents, and in wound dressings, it may be necessary or desirable to incorporate a permeation enhancer into the composition in order to enhance the rate of penetration of the agent into or through the skin. Suitable enhancers include, for example, the following: sulfoxides such as dimethylsulfoxide (DMSO) and decylmethylsulfoxide (C₁₀MSO); ethers such as diethylene glycol monoethyl ether (available commercially as Transcutol®) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80) and lecithin (U.S. Pat. No. 4,783,450); the 1-substituted azacycloheptan-2-ones, particularly 1-n-dodecylcyclaza-cycloheptan-2-one (available under the trademark Azone® from Nelson Research & Development Co., Irvine, Calif.; see U.S. Pat. Nos. 3,989,816, 4,316,893, 4,405,616 and 4,557,934); alcohols such as ethanol, propanol, octanol, decanol, benzyl alcohol, and the like; fatty acids such as lauric acid, oleic acid and valeric acid; fatty acid esters such as isopropyl myristate, isopropyl palmitate, methylpropionate, and ethyl oleate; polyols and esters thereof such as propylene glycol, ethylene glycol, glycerol, butanediol, polyethylene glycol, and polyethylene glycol monolaurate (PEGML; see, e.g., U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids, particularly salicylic acid and salicylates, citric acid and succinic acid. Mixtures of two or more enhancers may also be used.

Delivery Systems

The pharmaceutical compositions of this invention may be delivered to a patient using a variety of delivery systems. For instance, an active agent may be delivered to a body surface by simply placing a pharmaceutical composition of the invention on a body surface in active agent-transmitting relation thereto. Alternatively, an active agent-containing pharmaceutical composition may be incorporated into a delivery system or “patch.” In manufacturing such systems, the pharmaceutical adhesive composition may be cast or extruded onto a backing layer or release liner and will serve as the skin-contacting face of the system and act as an active agent reservoir. Alternatively, the pharmaceutical composition may be used as an active agent reservoir within the interior of such a system, with a conventional skin contact adhesive laminated thereto to affix the system to a patient's body surface.

Systems for the topical, transdermal or transmucosal administration of an active agent may comprise: (A) a reservoir containing a therapeutically effective amount of an active agent; (B) an adhesive means for maintaining the system in active agent transmitting relationship to a body surface; and (C) a backing layer as described in the preceding section, wherein (D) a disposable release liner covers the otherwise exposed adhesive, protecting the adhesive surface during storage and prior to use. In many such devices, the reservoir can also serve as the adhesive means, and the pharmaceutical compositions of the invention can be used as the reservoir and/or the adhesive means.

Any number of active agents can be administered using such delivery systems, as alluded to earlier herein. Suitable active agents include the broad classes of compounds normally delivered to and/or through body surfaces and membranes. With some active agents, it may be necessary to administer the agent along with a permeation enhancer in order to achieve a therapeutically effective flux through the skin, as also indicated previously.

Accordingly, a pharmaceutical composition can be incorporated into the reservoir, either during manufacture of the system or thereafter. The pharmaceutical composition will contain a quantity of an active agent effective to provide the desired dosage over a predetermined delivery period. The composition may also contain a carrier (e.g., a vehicle to solubilize the active agent), a permeation enhancer, if necessary, and optional excipients such as colorants, thickening agents, stabilizers, surfactants and the like. Other agents may also be added, such as antimicrobial agents, to prevent spoilage upon storage, i.e., to inhibit growth of microbes such as yeast and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof.

More than one reservoir may be present, each containing a different component for delivery into the skin.

The backing layer of the drug delivery system can function as the primary structural element of the transdermal system. The material used for the backing layer should be inert and may be incapable of absorbing drug, enhancer or other components of the pharmaceutical composition. Also, the material used for the backing layer should permit the device to follow the contours of the skin and be worn comfortably on areas of skin such as at joints or other points of flexure, that are normally subjected to mechanical strain with little or no likelihood of the device disengaging from the skin due to differences in the flexibility or resiliency of the skin and the device. Examples of materials useful for the backing layer are polyesters, polyethylene, polypropylene, polyurethanes and polyether amides. The layer is preferably in the range of about 15 microns to about 250 microns in thickness, and may, if desired, be pigmented, metallized, or provided with a matte finish suitable for writing. The layer is preferably although not necessarily nonocclusive (or “breathable”), i.e., is preferably permeable to moisture.

Additional layers, e.g., intermediate fabric layers and/or rate-controlling membranes, may also be present in a transdermal drug delivery system. Fabric layers may be used to facilitate fabrication of the device, while a rate-controlling membrane may be used to control the rate at which a component permeates out of the device. The component may be a drug, a permeation enhancer, or some other component contained in the drug delivery system.

The pharmaceutical compositions of the invention may also serve to deliver an active agent using other routes of administration. For example, the pharmaceutical compositions may be formulated with excipients, carriers, and the like suitable for oral administration of an orally active drug. The compositions may also be used in buccal and sublingual drug delivery, insofar as the compositions can adhere well to moist surfaces within the mouth. In buccal and sublingual systems, hydrolyzable, and/or bioerodible polymers may be incorporated into the compositions to facilitate gradual erosion throughout a drug delivery period. Still other types of formulations and drug delivery platforms may be prepared using the present compositions, including implants, rectally administrable compositions, vaginally administrable compositions, and the like.

In a still further embodiment of the invention, a delivery system is provided in the form of a flexible, laminated strip in which a pharmaceutical composition as described above, containing approximately 1.0 wt. % to 50.0 wt. %, preferably 1.0 wt. % to 30.0 wt. %, of at least one active agent, serves as an “interior,” body surface-contacting layer, and a second layer, adjacent to the body surface-contacting layer and comprised of a hydrophobic polymer containing 1.0 wt. % to 30.0 wt. %, preferably 1.0 wt. % to 10 wt. %, of at least one active agent, serves as the outer surface of the strip following application of the system to a body surface. The interior layer is capable of adhering to the body surface in the presence of moisture. In this embodiment, then, a drug delivery system is provided that includes two flexible, soft layers with differential permeability, the outer layer being measurably permeable but somewhat less permeable than the inner layer. Active agent is present in both layers, with the outer layer essentially serving as an additional reservoir for the agent(s). The outer layer is relatively hydrophobic (i.e., hydrophobic relative to the polymer(s) of the interior layer) such that the system is prevented from sticking to other body surfaces and releasing any significant amount of active agent onto the other body surface. The outer layer may also contain inert and/or active additives as described above with regard to the pharmaceutical composition per se. A particularly preferred polymer suitable as the primary component of the outer layer is Eudragit® RS-PO, which, as noted earlier herein, is a copolymer of neutral methacrylic acid esters and a small proportion of trimethylammonioethyl methacrylate.

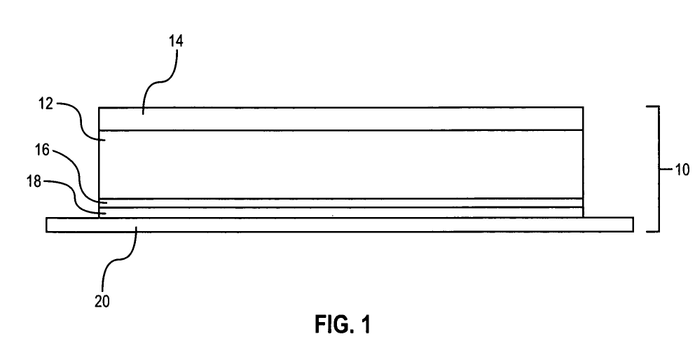

A representative drug delivery system of the invention is illustrated schematically in FIG. 1. The system 10 is composed of an interior layer bisected by a nonwoven layer 16, such that the active agent-containing reservoir includes an upper region 12 and a lower region 18. The upper region is laminated to the outer backing layer 14, composed of a relatively hydrophobic, permeable polymer and containing 1.0 wt. % to 30.0 wt. % active agent. Layer 14, as may be seen, provides the exterior surface of the system following application to the body surface. Removable release liner 20 covers the otherwise exposed surface of the lower region 18 of the system prior to use.

The pharmaceutical compositions of the invention are used by removing the product from its package, typically a moisture-free sealed pouch, removing the release liner, and applying the adhesive layer to the body surface. The delivery systems described herein can be provided in a variety of sizes, so that the composition can be applied to various different portions of body surfaces. The system can be left in place for an extended period of time, typically in the range of about 10 minutes to 8 hours, preferably in the range of about 30 to 60 minutes. The system can be readily removed by peeling it away from the body surface.

Conductive Compositions

The compositions of the invention can be rendered electrically conductive for use in biomedical electrodes and other electrotherapy contexts, i.e., to attach an electrode or other electrically conductive member to the body surface. For example, the present composition, formulated so as to exhibit pressure-sensitive adhesion, may be used to attach a transcutaneous nerve stimulation electrode, an electrosurgical return electrode, or an EKG electrode to a patient's skin or mucosal tissue. These applications involve modification of the pharmaceutical composition so as to contain a conductive species. Suitable conductive species are ionically conductive electrolytes, particularly those that are normally used in the manufacture of conductive adhesives used for application to the skin or other body surface, and include ionizable inorganic salts, organic compounds, or combinations of both. Examples of ionically conductive electrolytes include, but are not limited to, ammonium sulfate, ammonium acetate, monoethanolamine acetate, diethanolamine acetate, sodium lactate, sodium citrate, magnesium acetate, magnesium sulfate, sodium acetate, calcium chloride, magnesium chloride, calcium sulfate, lithium chloride, lithium perchlorate, sodium citrate and potassium chloride, and redox couples such as a mixture of ferric and ferrous salts such as sulfates and gluconates. Preferred salts are potassium chloride, sodium chloride, magnesium sulfate, and magnesium acetate, and potassium chloride is most preferred for EKG applications. Although virtually any amount of electrolyte may be present in the adhesive compositions of the invention, it is preferable that any electrolyte present be at a concentration in the range of about 0.1 to about 15 wt. % of the pharmaceutical composition. The procedure described in U.S. Pat. No. 5,846,558 to Nielsen et al. for fabricating biomedical electrodes may be adapted for use with the pharmaceutical compositions of the invention, and the disclosure of that patent is incorporated by reference with respect to manufacturing details. Other suitable fabrication procedures may be used as well, as will be appreciated by those skilled in the art.

Wound Dressings

In a preferred embodiment, the water-swellable, water-insoluble polymer compositions of the invention are used as absorbent materials in a wound dressing. In this embodiment, the water-swellable, water insoluble polymer compositions are prepared so that they are substantially nontacky, or at most slightly tacky, when applied to the body surface. The water-swellable, water insoluble polymer composition may be formulated so as to contain a pharmacologically active agent. Preferred active agents, in this embodiment, include the bacteriostatic and bactericidal agents, antibiotic agents, and pain-relieving agents set forth above, as well as the following:

Topical Vasodilators: Such compounds are useful for increasing blood flow in the dermis, and preferred topical vasodilators are those known as rubefacients or counterirritants. Rubefacient agents include nicotinic acid, nicotinates such as methyl, ethyl, butoxyethyl, phenethyl and thurfyl nicotinate, as well as essential oils such as mustard, turpentine, cajuput and capsicum oil, and components thereof. Particular preferred such compounds include, but are not limited to, methyl nicotinate, nicotinic acid, nonivamide, and capsaicin.

Proteolytic enzymes: Proteolytic enzymes herein are those that are effective wound cleansing agents, and include, for example, pepsin, trypsin, collagenase, chymotrypsin, elastase, carboxypeptidase, aminopeptidase, and the like.

Peptide, proteins, and amino acids: Suitable peptides and proteins are tissue-healing enhancing agents (also referred to in the art as “tissue regenerative agents”) such as collagen, glycosaminoglycans (e.g., hyaluronic acid, heparin, heparin sulfate, chondroitin sulfate, etc.), proteoglycans (e.g., versican, biglycan), substrate adhesion molecules (e.g., fibronectin, vitronectin and laminin), polypeptide growth factors (e.g., platelet-derived growth factor, a fibroblast growth factor, a transforming growth factor, an insulin-like growth factor, etc.), and other peptides such as osteopontin, and thrombospondin, all of which contain the tripeptide sequence RGD (arginine-glycine-aspartic acid), a sequence generally associated with adhesive proteins and necessary for interaction with cell surface receptors.

One embodiment of a wound dressing of the invention comprises an outer backing layer that serves as the external surface of the dressing following application to the body surface; a skin contact adhesive layer laminated thereto, which is an adhesive water-swellable, water insoluble polymer composition of the invention, optionally containing one or more pharmacologically active agents; an absorbent wound-contacting region comprised of a water-swellable, water insoluble polymer composition of the invention and located on the on the wound contacting side of layer; and a removable release liner. Upon removal of the release liner, the dressing is applied to a body surface in the region of a wound, and placed on the body surface so that the wound-contacting region is directly over the wound. In this embodiment, the wound dressing adheres to the skin surrounding the wound as a result of the exposed skin contact adhesive areas surrounding the wound-contacting region. If the wound-contacting water-swellable, water insoluble polymer composition is prepared so that it has some degree of tack prior to absorption of water (as in, e.g., wound exudate), the dressing adheres in the central region as well. It should be noted that any of the water-swellable, water insoluble polymer compositions of the invention may be used as a wound dressing herein, providing that, as noted above, the water-swellable, water insoluble polymer composition is substantially nontacky or at most slightly tacky. Also, those water-swellable, water insoluble polymer compositions that exhibit a high degree of absorbency are preferred.

In this embodiment, the backing layer of the wound dressing functions as the primary structural element and provides the dressing with flexibility. The material used for the backing layer should be inert, and should permit the device to follow the contours of the skin and be worn comfortably on areas of skin such as at joints or other points of flexure, that are normally subjected to mechanical strain with little or no likelihood of the device disengaging from the skin due to differences in the flexibility or resiliency of the skin and the device. Examples of materials useful for the backing layer are polyesters, polyethylene, polypropylene, polyurethanes and polyether amides. The layer is preferably in the range of about 15 microns to about 250 microns in thickness, and may, if desired, be pigmented, metallized, or provided with a matte finish suitable for writing. The layer is preferably although not necessarily nonocclusive (or “breathable”), i.e., is preferably permeable to moisture.

The release liner is a disposable element that serves to protect the device prior to application. The release liner should be formed from a material impermeable to the drug, vehicle and adhesive, and that is easily stripped from the adhesive. Release liners are typically treated with silicone or fluorocarbons, and are commonly made from polyesters, polyethylene, and polyethylene terephthalate.

In another embodiment, the backing layer of the wound dressing is composed of a tacky or at least slightly tacky water-swellable, water insoluble polymer composition of the invention, but is provided with a nontacky upper surface. The wound-contacting water-swellable, water insoluble polymer material is adhered to the skin-contacting side of the backing layer. Upon removal of release liner, the wound dressing is applied to an individual's skin in the region of a wound so that the wound-contacting water-swellable, water insoluble polymer material is placed directly over the wound. As with the previous embodiment, the wound dressing adheres to the body surface by virtue of the exposed regions of the adhesive water-swellable, water insoluble polymer composition. In this case, it is preferred that both the backing layer and the water-swellable, water insoluble polymer be translucent, so that the extent of wound healing can be viewed directly through the backing, eliminating the need for frequent replacement or removal of the wound dressing.

In a further embodiment, the perimeter of the wound dressing is made of a different material than the interior region of the backing. In this case, the perimeter is comprised of a skin contact adhesive that may or may not be an adhesive water-swellable, water insoluble polymer composition of the invention, although the upper, outwardly facing surface of the perimeter is nontacky. The interior region of the backing is preferably comprised of a water-swellable, water insoluble polymer composition of the invention. The skin-facing side of the interior region may or may not be tacky, although the upper surface of the interior region should be nontacky. The wound-contacting water-swellable, water insoluble polymer material is adhered to the underside (i.e., the skin contacting side) of the backing and is centrally located within interior region. As with the previous embodiment, it is preferred that both the interior region of the backing and the wound-contacting water-swellable, water insoluble polymer material are translucent. Generally, the perimeter adhesive will be opaque.

In a variation on the previous embodiment, an outer layer may be laminated to the upper surface of the device shown. Such an outer layer would then serve as the actual backing.

In still another embodiment, the wound dressing contains three layers, a backing layer, a central adhesive layer typically composed of a conventional pressure-sensitive adhesive, and a wound-contacting water-swellable, water insoluble polymer layer, wherein the three layers are coextensive such that there is no distinct perimeter region as there is in the previous embodiments. During storage and prior to use, the skin contacting side of the dressing is protected with a release liner (not shown), as above.

This last embodiment can be varied such that the wound dressing is composed of only two layers, a backing and a wound-contacting water-swellable, water insoluble polymer layer laminated thereto and coextensive therewith. In this case, the water-swellable, water insoluble polymer layer must have sufficient tack so as to adhere to the backing layer, even after water absorption. As with the embodiments discussed above, the skin contacting side is protected with a release liner during storage and prior to use.

Optional Additives

The adhesive compositions of the invention may also include one or more conventional additive, which may be combined with the polymers and the plasticizer during adhesive formulation, or incorporated thereafter. Optional additives include, without limitation, fillers, pH regulating agents, ionizing agents, tackifiers, detackifying agents, electrolytes, antimicrobial agents, antioxidants, preservatives, colorants, flavors, and combinations thereof.

In certain embodiments, the compositions of the invention may also include a pharmacologically active agent or a cosmeceutically active agent. For instance, transdermal, transmucosal, and topical delivery systems in which an adhesive composition of the invention serves as a drug reservoir and/or skin contact adhesive layer, may be formulated for the delivery of a specific pharmacologically active agent. Cosmeceutical products such as face masks and eye pads may include active agents for treating skin.

Absorbent fillers may be advantageously incorporated to control the degree of hydration when the adhesive is on the skin or other body surface. Such fillers can include microcrystalline cellulose, talc, lactose, kaolin, mannitol, colloidal silica, alumina, zinc oxide, titanium oxide, magnesium silicate, magnesium aluminum silicate, hydrophobic starch, calcium sulfate, calcium stearate, calcium phosphate, calcium phosphate dihydrate, woven and non-woven paper, and cotton materials. Other suitable fillers are inert, i.e., substantially non-adsorbent, and include, for example, polyethylenes, polypropylenes, polyurethane polyether amide copolymers, polyesters and polyester copolymers, nylon and rayon. A preferred filler is colloidal silica, e.g., Cab-O-Sil® (Cabot Corporation, Boston, Mass.).

Compounds useful as pH regulators include, but are not limited to, glycerol buffers, citrate buffers, borate buffers, phosphate buffers, and citric acid-phosphate buffers. Buffer systems are useful to ensure, for instance, that the pH of a composition of the invention is compatible with that of an individual's body surface.

Ionizing agents are also useful to impart a desired degree of ionization to the interpolymer complex within the adhesive compositions of the invention. Suitable ionizing agents are acids and bases, depending on the group to be ionized. The acids and bases may be inorganic (hydrochloric acid, hydrobromic acid, sodium hydroxide, potassium hydroxide, sodium carbonate, ammonium carbonate, etc.) or organic (acetic acid, maleic acid, triethylamine, ethanolamine, etc.).

Tackifiers can also be included to improve the adhesive and tack properties of the compositions of the invention. The mechanism underlying tack improvement results from the large size and hydrophobic character of tackifier molecules. Exemplary tackifying materials include tacky rubbers such as polyisobutylene, polybutadiene, butyl rubber, polystyrene-isoprene copolymers, polystyrene-butadiene copolymers, and neoprene (polychloroprene). Other examples of suitable tackifiers herein are those that are conventionally used with pressure sensitive adhesives, e.g., rosins, rosin esters, polyterpenes, and hydrogenated aromatic resins. In those embodiments wherein adhesion is to be reduced or eliminated, conventional detackifying agents may also be used. Suitable detackifiers include, but are not limited to, crosslinked poly(vinylpyrrolidone), silica gel, and bentonites.

Preferred thickeners for the water-swellable, water-insoluble polymers and systems herein are naturally occurring compounds or derivatives thereof, and include, by way of example: collagen; galactomannans; starches; starch derivatives and hydrolysates; cellulose derivatives such as methyl cellulose, hydroxypropylcellulose, hydroxyethyl cellulose, and hydroxypropyl methyl cellulose; colloidal silicic acids; and sugars such as lactose, saccharose, fructose and glucose. Synthetic thickeners such as polyvinyl alcohol, vinylpyrrolidone-vinylacetate-copolymers, polyethylene glycols, and polypropylene glycols may also be used.

As discussed above, the compositions of the invention can be rendered electrically conductive for use in biomedical electrodes and other electrotherapy contexts, i.e., to attach an electrode or other electrically conductive member to the body surface. For example, the composition may be used to attach a transcutaneous nerve stimulation electrode, an electrosurgical return electrode, or an EKG electrode to a patient's skin or mucosal tissue. These applications involve modification of the composition so as to contain a conductive species. Suitable conductive species are ionically conductive electrolytes, particularly those that are normally used in the manufacture of conductive adhesives used for application to the skin or other body surface, and include ionizable inorganic salts, organic compounds, or combinations of both. Examples of ionically conductive electrolytes include, but are not limited to, ammonium sulfate, ammonium acetate, monoethanolamine acetate, diethanolamine acetate, sodium lactate, sodium citrate, magnesium acetate, magnesium sulfate, sodium acetate, calcium chloride, magnesium chloride, calcium sulfate, lithium chloride, lithium perchlorate, sodium citrate and potassium chloride, and redox couples such as a mixture of ferric and ferrous salts such as sulfates and gluconates. Preferred salts are potassium chloride, sodium chloride, magnesium sulfate, and magnesium acetate, and potassium chloride is most preferred for EKG applications. Although virtually any amount of electrolyte may be present in the adhesive compositions of the invention, it is preferable that any electrolyte present be at a concentration in the range of about 0.1 to about 15 wt. % of the hydrogel composition. The procedure described in U.S. Pat. No. 5,846,558 to Nielsen et al. for fabricating biomedical electrodes may be adapted for use with the hydrogel compositions of the invention, and the disclosure of that patent is incorporated by reference with respect to manufacturing details. Other suitable fabrication procedures may be used as well, as will be appreciated by those skilled in the art.

Antimicrobial agents may also be added to the compositions of the invention. Antimicrobial agents function by destroying microbes, preventing their pathogenic action, and/or inhibiting their growth. Desirable properties of antimicrobial agents include, but are not limited to: (1) the ability to inactivate bacteria, viruses and fungi, (2) the ability to be effective within minutes of application and long after initial application, (3) cost, (4) compatibility with other components of composition, (5) stability at ambient temperature, and (6) lack of toxicity.

Antioxidants may be incorporated into the compositions of the invention in lieu of or in addition to any antimicrobial agent(s). Antioxidants are agents that inhibit oxidation and thus prevent the deterioration of preparations by oxidation. Suitable antioxidants include, by way of example and without limitation, ascorbic acid, ascorbyl palmitate, butylated hydroxyanisole, butylated hydroxytoluene, hypophophorous acid, monothioglycerol, sodium ascorbate, sodium formaldehyde sulfoxylate and sodium metabisulfite and others known to those of ordinary skill in the art. Other suitable antioxidants include, for example, vitamin C, butylated hydroxytoluene (BHT), butylated hydroxyanisole (BHA), sodium bisulfite, vitamin E and its derivatives, propyl gallate, sulfite derivatives, and others known to those of ordinary skill in the art.

Other preservatives that can be incorporated into the present compositions include, by way of example, p-chloro-m-cresol, phenylethyl alcohol, phenoxyethyl alcohol, chlorobutanol, 4-hydroxybenzoic acid methylester, 4-hydroxybenzoic acid propylester, benzalkonium chloride, cetylpyridinium chloride, chlorohexidine diacetate or gluconate, ethanol, and propylene glycol.

The practice of the present invention will employ, unless otherwise indicated, conventional techniques of polymer chemistry, adhesive manufacture, and drug delivery, which are within the skill of the art. Such techniques are fully explained in the literature.

It is to be understood that while the invention has been described in conjunction with the preferred specific embodiments, the description and examples that are presented above are intended to illustrate and not limit the scope of the invention. Other aspects, advantages and modifications will be apparent to those skilled in the art to which the invention pertains. All patents, patent applications, journal articles, and other references cited herein are incorporated by reference in their entireties.

Fabrication

The water-swellable, water-insoluble polymer compositions of the invention are generally melt extrudable, and thus may be prepared using a simple blending and extruding process. The components of the composition are weighed out and then admixed, for example using a Brabender or Baker Perkins Blender, generally although not necessarily at an elevated temperature, e.g., about 90° C. to about 140° C. The resulting formulation can be extruded using a single or twin extruder, or pelletized. Preferably the formulation is extruded directly onto a substrate such as a backing layer or release liner, and then pressed. In a particularly preferred embodiment, the formulation is extruded onto an outer layer composed of a permeable polymer matrix. The thickness of the resulting laminate will be in the range of about 0.05 mm to about 0.80 mm, more usually in the range of about 0.1 mm to about 0.25 mm. Other manufacturing processes, e.g., solvent casting as described in No. US 2003/0152528 A1 to Singh et al. can also be employed.

Optimized Compositions

In a preferred embodiment, a water-swellable, water-insoluble polymer composition is provided that is composed of an admixture of: 1.5 wt. % to 30 wt. %, preferably 1.5 wt. % to 20 wt. %, more preferably 1.5 wt. % to 90 wt. %, and most preferably 1.5 wt. % to 95 wt. %, of a hydrophilic polymer composition composed of (a) a covalently crosslinked hydrophilic polymer, and/or (b) a blend of a hydrophilic polymer and a complementary oligomer capable of hydrogen bonding thereto; 40 wt. % to 90 wt. %, preferably 45 wt. % to 90 wt. %, more preferably 50 wt. % to 90 wt. %, and most preferably 60 wt. % to 90 wt. %, of at least one water-swellable, water-insoluble polymer; and at least one active agent.

We claim:

1. A pharmaceutical composition comprising an admixture of:
a therapeutically effective amount of an active agent; and
at least two water-swellable, water-insoluble polymers that in combination render the composition adhesive upon contact with moisture, wherein a first water-swellable, water-insoluble polymer is cationic, a second water-swellable, water-insoluble polymer is anionic, and the polymers are ionically associated with each other to form a polymer matrix.

2. The pharmaceutical composition of claim 1, wherein at least one of the water-swellable, water-insoluble polymers is an acrylate-based polymer.

3. The pharmaceutical composition of claim 2, wherein the acrylate-based polymer is a polymer or copolymer of acrylic acid, methacrylic acid, acrylate, methyl acrylate, ethyl acrylate, methyl methacrylate, ethyl methacrylate, a dialkylaminoalkyl acrylate, a dialkylaminoalkyl methacrylate, a trialkylammonioalkyl acrylate, and/or a trialkylammonioalkyl methacrylate.

4. The pharmaceutical composition of claim 3, wherein the acrylate-based polymer is a polymer or copolymer of acrylic acid, methacrylic acid, methyl methacrylate, ethyl methacrylate, 2-dimethylaminoethyl methacrylate, and/or trimethylammonioethyl methacrylate chloride.

5. The pharmaceutical composition of claim 1, wherein the water-swellable, water-insoluble polymers are acrylate-based polymers.

6. The pharmaceutical composition of claim 5, wherein the acrylate-based polymers are polymers or copolymers of acrylic acid, methacrylic acid, acrylate, methyl acrylate, ethyl acrylate, methyl methacrylate, ethyl methacrylate, a dialkylaminoalkyl acrylate, a dialkylaminoalkyl methacrylate, a trialkylammonioalkyl acrylate, and/or a trialkylammonioalkyl methacrylate.

7. The pharmaceutical composition of claim 6, wherein the acrylate-based polymers are polymers or copolymers of acrylic acid, methacrylic acid, methyl methacrylate, ethyl methacrylate, 2-dimethylaminoethyl methacrylate, and/or trimethylammonioethyl methacrylate chloride.

8. The pharmaceutical composition of claim 1, wherein the cationic polymer is an acrylate-based polymer with pendant quaternary ammonium groups, and the anionic polymer is an ionized acrylic acid or methacrylic acid polymer.

9. The pharmaceutical composition of claim 1, further comprising 1.5 wt. % to 30 wt. % of a crosslinked hydrophilic polymer composition composed of (a) a covalently crosslinked hydrophilic polymer, and/or (b) a blend of a hydrophilic polymer and a complementary oligomer capable of hydrogen bonding thereto.

10. The pharmaceutical composition of claim 9, wherein the crosslinked hydrophilic polymer composition represents up to about 10 wt. % of the pharmaceutical composition.

11. The pharmaceutical composition of claim 9, wherein the water-swellable, water-insoluble polymers represent at least 60 wt. % of the pharmaceutical composition.

12. The pharmaceutical composition of claim 9, wherein:
the hydrophilic polymer is selected from poly(N-vinyl lactams), poly(N-vinyl amides), poly(N-alkylacrylamides), polyvinyl alcohol, polyvinylamine, and copolymers thereof; and
the complementary oligomer is selected from polyalcohols, monomeric and oligomeric alkylene glycols, polyalkylene glycols, carboxyl-terminated polyalkylene glycols, amino-terminated polyalkylene glycols, ether alcohols, alkane diols, and carbonic diacids.

13. The pharmaceutical composition of claim 10, wherein:
the hydrophilic polymer is a poly(N-vinyl lactam); and
the complementary oligomer is selected from the group consisting of polyethylene glycol and carboxyl-terminated polyethylene glycol.

14. The pharmaceutical composition of claim 1, further comprising a crosslinked hydrophilic polymer.

15. A delivery system for topical or transdermal administration of a pharmacologically active agent, comprising a laminated composite of:
a skin contact adhesive layer comprising the pharmaceutical composition of claim 1; and,
laminated thereto, a flexible backing material that serves as the outer surface of the system following application to a body surface.

16. The delivery system of claim 15, further including a removable release liner covering the skin contact adhesive layer prior to use, said release liner preventing exposure of the layer to air.

17. The delivery system of claim 15, wherein the flexible backing material is comprised of a hydrophobic polymer.

18. The delivery system of claim 17, wherein the flexible backing material is permeable.

19. The delivery system of claim 15, wherein the skin contact adhesive layer is bisected into two separate layers by a nonwoven layer.

20. A packaged, anhydrous active agent delivery system, comprising the delivery system of claim 15 in a moisture-free sealed pouch.

21. A conductive bioadhesive composition, comprising:
at least two water-swellable, water-insoluble polymers that in combination render the composition adhesive upon contact with moisture, wherein a first water-swellable, water-insoluble polymer is cationic, a second water-swellable, water-insoluble polymer is anionic, and the polymers are ionically associated with each other to form a polymer matrix; and
an amount of an ionically conductive electrolyte effective to render the composition electrically conductive.

22. The conductive bioadhesive composition of claim 21, wherein the ionically conductive electrolyte is selected from ionizable inorganic salts, organic compounds, and combinations thereof.

23. A conductive bioadhesive composition of claim 21, wherein the ionically conductive electrolyte is selected from ammonium sulfate, ammonium acetate, monoethanolamine acetate, diethanolamine acetate, sodium lactate, sodium citrate, magnesium acetate, magnesium sulfate, sodium acetate, calcium chloride, magnesium chloride, calcium sulfate, lithium chloride, lithium perchlorate, sodium citrate and potassium chloride, redox couples, potassium chloride, sodium chloride, magnesium sulfate, magnesium acetate, and combinations thereof.

24. The conductive bioadhesive composition of claim 21, wherein an electrolyte is present at a concentration in the range of about 0.1 to about 15 wt. % of the conductive bioadhesive.

25. A wound dressing comprising a laminated composite of a body facing layer having a body-contacting surface, and an outwardly facing backing layer, wherein at least a portion of the body-contacting surface is composed of a water-swellable, water-insoluble polymer composition comprising at least two water-swellable, water-insoluble polymers that in combination render the composition adhesive upon contact with moisture, wherein a first water-swellable, water-insoluble polymer is cationic, a second water-swellable, water-insoluble polymer is anionic, and the polymers are ionically associated with each other to form a polymer matrix.

26. The wound dressing of claim 25, wherein the entire body-contacting surface is comprised of the water-swellable, water-insoluble polymer composition.

27. The wound dressing of claim 26, wherein the body-facing layer has a perimeter comprised of a skin-contact adhesive and an inner region containing the water-swellable, water-insoluble polymer composition.

28. The wound dressing of claim 27, wherein a central, wound-contacting portion of the inner region is comprised of the water-swellable, water-insoluble polymer composition.

29. The wound dressing of claim 25, wherein the backing layer is nonocclusive.

30. The wound dressing of claim 25, wherein the backing layer is occlusive.

31. The wound dressing of claim 25, further including a pressure-sensitive adhesive layer between the body-facing layer and the backing layer.

32. The wound dressing of claim 25, further including a removable release liner covering and co-extensive with the body-facing surface.

33. The wound dressing of claim 25, further including an active agent suitable for application to a wound.

34. The wound dressing of claim 33, wherein the active agent is selected from the group consisting of bacteriostatic and bactericidal compounds, antibiotic agents, pain relieving agents, topical vasodilators, tissue-healing enhancing agents, amino acids, proteins, proteolytic enzymes, cytokines, and polypeptide growth factors.
